(19) United States
(12) Patent Application Publication
(10) Pub. No.: US 2003/0083746 A1
(43) Pub. Date: May 1, 2003

(54) Vertebral spacer for spinal stabilization

(76) Inventor: Stephen D. Kuslich, Stillwater, MN (US)

Correspondence Address: VIDAS, ARRETT & STEINKRAUS, P.A., 6109 BLUE CIRCLE DRIVE, SUITE 2000, MINNETONKA, MN, 55343-9185, US

(21) Appl. No.: 10/000,769

(22) Filed: Oct. 31, 2001

(51) Int. Cl. A61F 2/44; A61F 2/28

(52) U.S. Cl. 623/17.11; 623/23.49; 606/61

(57) ABSTRACT

A device for implantation into a mammalian spine which comprises: a body having a first end and a second end. The first end constructed and arranged to engage a lower surface of a spinal body when the body is implanted into an intervertebral space within the mammalian spine. The second end constructed and arranged to engage an upper surface of a spinal body when the body is implanted into an intervertebral space of the mammalian spine. The body defines an interior space. The body further includes a first edge and a second edge which define a longitudinal discontinuity extending from the first end of the body to the second end of the body, wherein the longitudinal discontinuity may be a space, slot or groove.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

Certain diseases and injuries damage the spinal tissues. This damage may lead to spinal instability. Instability of the spine may cause pain, and/or mal-alignment, and/or neurological impairment. Surgeons frequently treat these conditions with operations designed to correct these instabilities and mal-alignments. These operations generally involve implanting fixation devices to re-align and/or restabilize the spine.

2. Description of the Related Art

In repairing a damaged portion or portions of a spine or spinal body, total or sub-total removal of the vertebra and/or discs may be necessary to decompress the spinal canal when disease and/or injury causes massive destruction of the spinal bones and the surrounding discs. Reconstruction of the resulting defect is necessary to regain spinal stability. Devices used to reconstruct such defects are commonly referred to as interbody spacers, spinal cages, spinal baskets, or corpectomy devices.

Devices currently available range from large bone grafts to metallic or ceramic tubes (hollow or solid) that span the defect. Examples include femoral or iliac allograft or autograft, or turnbuckle devices such as in U.S. Pat. No. 4,401,112, or porous metal tubes such as the jacket of U.S. Pat. No. 4,820,305, or devices such as U.S. Pat. No. 403,069.

Other devices are disclosed in U.S. Pat. Nos. 5,405,391; 5,735,899; 5,571,189; 5,591,235; 6,086,589; 5,549,679; and 6,056,749. The entire content of all patents and patent applications recited herein being incorporated in their entirety herein by reference.

Many of the devices presently available, particularly cage-type spacers, are difficult to place, or utilize at all, with a previously positioned intermedullary rod. With many of these devices, it is also difficult, or impossible, to place an intermedullary rod after a cage-type spacer is inserted. A specific example further defines the problem:

If it is desired by a surgeon to insert both a Harms-type cage (U.S. Pat. No. 4,820,305) and the intermedullary rod portion of the K-Centrum System (U.S. Pat. No. 5,591,235), the rod must first be placed into the interior portion of the cage. Then the cage plus the rod must be simultaneously placed into the defect being treated. Simultaneously placing both devices can be awkward, time consuming and dangerous. Many surgeons would prefer to place the cage into position first, followed by rod insertion, because once the cage is in position, external distraction devices can be removed, allowing better visualization of the operative site. But, if the cage has already been placed into position, it is nearly impossible to place the intermedullary rod without first cutting a slot or otherwise damaging the cage by indenting the case to form a deep groove.

Embodiments of the present invention address the problem associated with previous cage-type spacer designs by providing at least one type of cage having a slot, groove or opening that can accommodate the insertion or receipt of a rod therein. In the present invention the cage may also be placed on or about an intermedullary rod already in place within or adjacent to a spinal body or bodies. The present invention is also directed to various manners of construction and uses of cages having slots, grooves or openings designed to accommodate an intermedullary rod therein.

All US patents and applications and all other published documents mentioned anywhere in this application are incorporated herein by reference in their entirety.

The art described in this section is not intended to constitute an admission that any patent, publication or other information referred to herein is “prior art” with respect to this invention, unless specifically designated as such. In addition, this section should not be construed to mean that a search has been made or that no other pertinent information as defined in 37 C.F.R. '1.56(a) exists.

The invention in various of its embodiment is summarized below. Additional details of the invention and/or additional embodiments of the invention may be found in the Detailed Description of the Invention below.

The abstract provided herewith is intended to comply with 37 CFR 1.72 and is not intended be used in determining the scope of the claimed invention.

### BRIEF SUMMARY OF THE INVENTION

The present invention is directed to a variety of different embodiments. In at least one embodiment the invention relates to a novel device for use in stabilizing and/or realigning a portion of a mammalian spine. More specifically, the present invention is directed to various embodiments of a vertebral spacer that provides support for the spine.

In at least one embodiment, the present invention is directed to a device that facilitates bone union between two adjacent or non-adjacent vertebrae. The device provides a load bearing and/or load sharing container that can be filled with bone graft or bone graft substitutes, with or without the addition of bone-growth stimulating chemicals such as bone morphogenic protein or other chemical or biological compounds, agents or gene therapy substances.

In at least one embodiment, the invention relates to a means for structuring a vertebral spacer in such a manner that the device can be easily fitted with an intermedullary rod, without the need to perform in-situ cutting or deforming the spacer, as is needed with current spacers.

In some embodiments, a slotted or grooved tubular device is configured to replace a vertebral body or a portion thereof, or an intervertebral disc, or any number of vertebral bodies and their adjacent spinal discs. Because of its unique construction, embodiments of the present invention may be configured to fit a spinal defect or vertebral space, that is to say, the space previously occupied by the injured vertebral body and/or the adjacent discs, defined by the inferior end plate of an upper vertebra and the superior end plate of a lower vertebra, without injurious contact with surrounding tissue structures, such as the spinal cord or the great vessels.

Embodiments of the invention may be constructed from any biocompatible material that has sufficient rigidity and strength to at least temporarily withstand the loads imposed by body weight and the forces exerted by muscles, ligaments and tendons.

In at least one embodiment of the invention the device may be fitted with transversely placed end support plates at its proximal and/or distal ends. These end support plates may be fitted with bone engaging teeth or other projections designed to grip the adjacent bony end plates.

In at least one embodiment of the invention, the end support plates have one or more protrusions that frictionally engage the adjacent vertebra.

In at least one embodiment of the device, the end support plates may contain posts that either impales the vertebral end plates above and below, or the posts may be affixed to bone anchors that have been placed on the vertebral bodies. Examples of such bone anchors include the K-Centrum™ bone anchor available from Spineology, Inc.

In at least one embodiment of the invention, the end support plates contain one or more openings.

In at least one embodiment of the present device, its proximal and distal ends are constructed so that they match the geometry of the end plates of the vertebral bodies above and below. These ends may also be perforated (or porous).

In at least one embodiment of the invention, end caps are placed on the proximal or distal ends of the device. The end caps may be stacked and/or linked increasing the length of the device incrementally to provide a custom fit to the specific vertebral space. The end caps may be flat and/or angled to accommodate any angulations of the spine. The end caps may be “u” shaped providing extra rigidity to the device. The end caps may have a wider rim than the device, providing increased surface area for the vertebral endplates to rest upon. The end caps may be fitted with ridges, knarling or other bone engaging projections to grip adjacent vertebral endplates.

In at least one embodiment of the invention, the device is constructed to allow trimming to fit a desired vertebral space or defect.

In at least one embodiment of the invention the device consists of a thin walled tube, wherein the tube wall is longitudinally deformed and displaced inward so that its transverse (cross sectional) shape contains a “u” shaped component that extends from the open section on the peripheral surface of the tube, to the base of the “u” shaped component lying somewhere interior to the tangential surface of the outside of the tube.

In at least one embodiment of the invention the device consists of a thin walled tube, wherein the space within the walls is empty. Bone graft or bone graft substitute with or without bone growth stimulating chemicals may be inserted into the empty space. The empty space within the walls of the device may be filled before and/or after the device has been inserted.

In at least one embodiment of the invention the ends and/or sides of the present device may be perforated to allow the in growth of blood vessel into the graft material contained within.

In at least one embodiment, the inventive device may be used as a standalone device or it may be combined with other forms of fixation. At least one embodiment is structured so that the device's geometry is designed to fit around an intermedullary rod that has been positioned in the space between two adjacent or non-adjacent vertebral bodies. In this regard, for instance, the device may be placed adjacent to, and around an existing patented spinal fixator such as the K-Centrum System, described in U.S. Pat. No. 5,591,235, the entire contents of which being incorporated herein by reference. Alternatively, the device could be positioned around any other form of spinal intermedullary rod or pin, such as a Harrington rod, or a simple Steinman pin. In at least one embodiment, the inventive device may be placed in the vertebral space followed by the insertion of a rod or pin and/or spinal fixator.

Further aspects of the invention may become apparent form the detailed description which follows.

### BRIEF DESCRIPTION OF THE SEVERAL VIEWS OF THE DRAWINGS

A detailed description of the invention is hereafter described with specific reference being made to the drawings in which:

FIG. 1 is a side elevational view of an embodiment of the invention shown in place within a mammalian spine.

FIG. 2 is a perspective view of an embodiment of the invention.

FIG. 3 is a top-down view of the embodiment of the invention shown in FIG. 2.

FIG. 4 is a perspective view of an embodiment of the invention.

FIG. 5 is a top-down view of the embodiment of the invention shown in FIG. 4.

FIG. 6 is a perspective view of an embodiment of the invention.

FIG. 7 is a top-down view of the embodiment of the invention shown in FIG. 6.

FIG. 8 is a perspective view of an embodiment of the invention wherein the device is shown in functional relationship with an intermedullary rod.

FIG. 9 is a perspective view of an embodiment of the invention wherein the interior of the device is shown containing fill material.

FIG. 10 is a partially exploded perspective view of an embodiment of the invention having end caps.

FIG. 11 is a top-down view of the end cap shown in FIG. 10

FIG. 12 is a perspective view of an end cap suitable for use with the embodiment shown in FIGS. 6 and 7.

FIG. 13 is a top-down view of the end cap shown in FIG. 12.

FIG. 14 is a side elevational view of the embodiment shown in FIG. 10 shown in place within a mammalian spine.

FIG. 15 is a side elevational view of an embodiment of the invention shown with an end cap mounted thereon.

FIG. 16 is a side elevational view of an embodiment of the invention shown with an end cap engaged thereto.

FIG. 17 is a side elevational view of an embodiment of the invention shown in place within a mammalian spine.

### DETAILED DESCRIPTION OF THE INVENTION

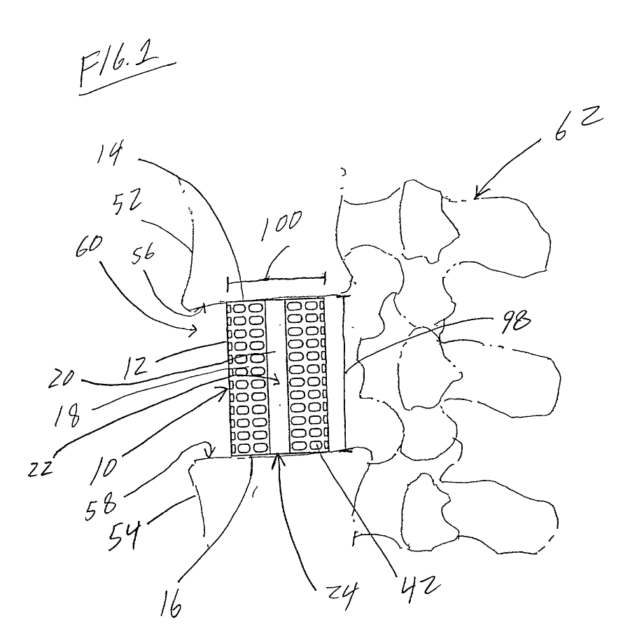

As may be seen in FIG. 1, the device 10 consists of a body 12 having a first end 14 and a second end 16. The body 12, has an exterior surface 18 and an inside surface 20 which defines an interior space 22. The body 12 further defines a longitudinal discontinuity 24 which extends from the first end 14 to the second end 16.

As previously indicated, device 10 is constructed and arranged to be inserted within an intervertebral space 60 of a mammalian spine 62 to assist in supporting, stabilizing and/or realigning a portion thereof. As may be seen in the embodiment shown in FIG. 1, ends 14 and 16 are designed to contact and/or be engaged by spinal bodies 52 and 54. Device 10 is inserted between two spinal bodies 52 and 54 in such a manner so as to ensure that end 14 engages the lower surface 56 of spinal body 52, and end 16 engages the upper surface 58 of spinal body 54. As a result the longitudinal discontinuity 24 is vertically or longitudinally oriented between the spinal bodies 52 and 54.

Spinal bodies 52 and 54 may be adjacent or non-adjacent vertebrae or they may be intervertebral discs or portions thereof.

As previously stated, Device 10 is intended to be inserted into an intervertebral space 60, the various dimensions of the body 12 should therefore correspond to the intended use. The body may have the generally cylindrical cross-sectional shape shown in FIGS. 3, 5 and 7 or the body 12 may have any cross-sectional shape or polygonal geometry as desired. Some specific shapes in which body 12 may be embodied include but are not limited to, substantially: triangular, rectangular, square, rhomboid, hexagonal, or pentagonal. Body 12 should have a length 98 of about 10 mm to about 150 mm, and more preferably about 15 mm to about 130 mm. The diameter 100 of device 12 should be about 5 mm to about 40 mm, and more preferably between 10 mm and about 35 mm.

The characteristics of the longitudinal discontinuity 24 will depend in-part, on the dimensions of the body 12, as well as on the size and shape of any intermedullary rod that may be potentially inserted therein. The longitudinal discontinuity 24 may be characterized in at least three different manners:

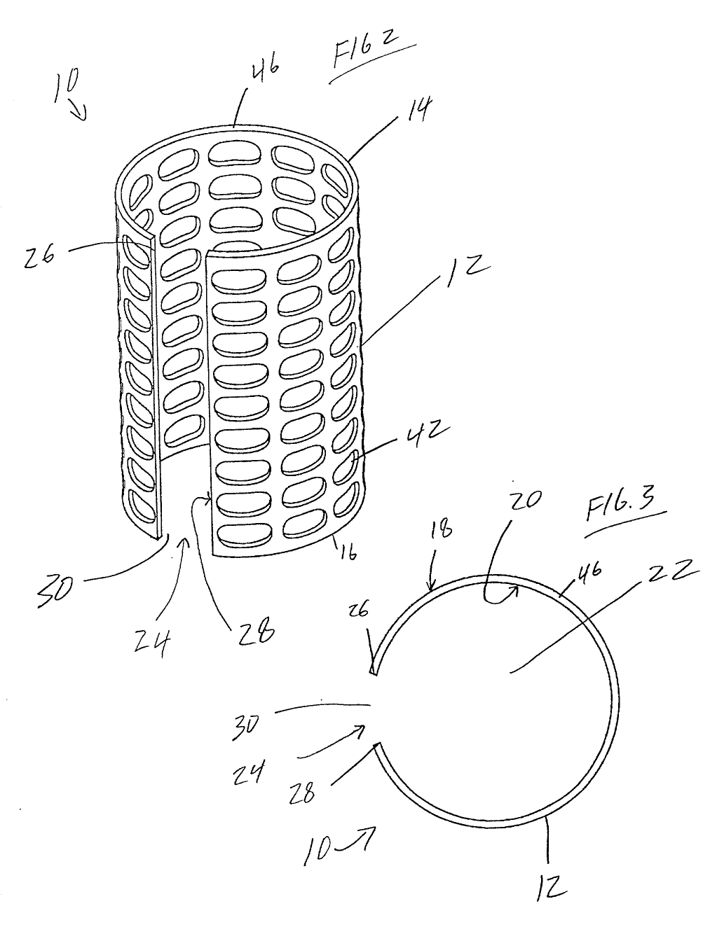

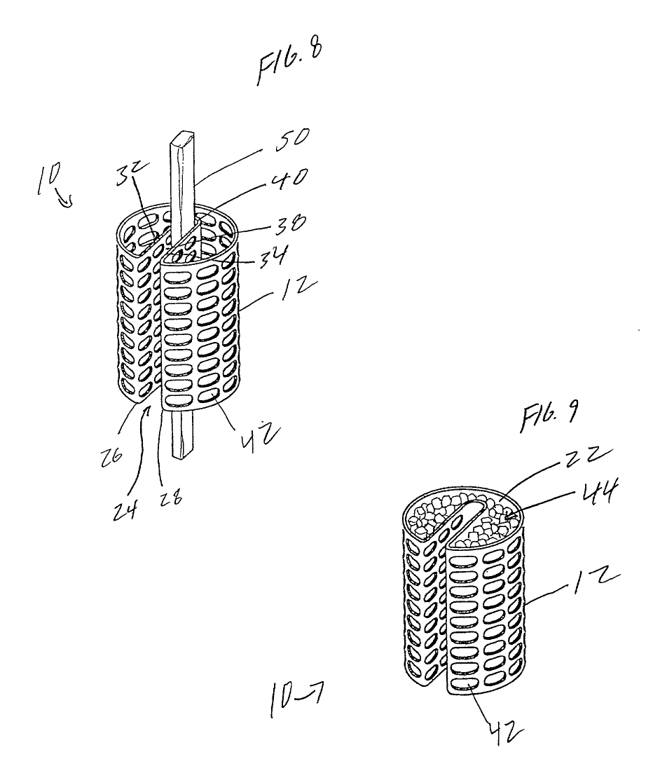

In the embodiments shown in FIGS. 2 and 3, the discontinuity 24 may be characterized as a longitudinal opening 30 defined by a first edge 26 and a second edge 28 of the body 12. The opening 30, is sized to allow an intermedullary rod 50 (shown in FIG. 8) to pass longitudinally therethrough. Where the discontinuity 24 is an opening 30, the edges 26 and 28 may be pressed together, spread apart or otherwise modified, prior to or subsequent to implantation to reduce or expand the size of opening 30 as desired.

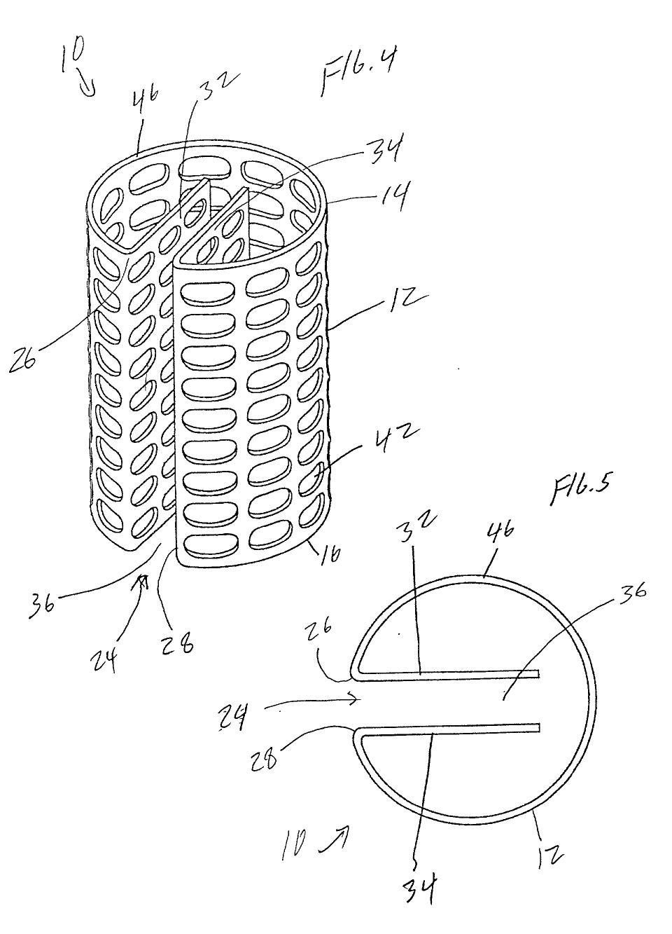

In a second embodiment shown in FIGS. 4 and 5 the device 10 includes a first guide wall 32 extends inwardly from the edge 26 and a second guide wall 34 extends inwardly from the edge 28 to form a slot 36 into which an intermedullary rod 50 (shown in FIG. 8) may be positioned. Walls 32 and 34 are preferably parallel. Walls 32 and 34 act to guide a rod or other member into the slot 36. The walls 32 and 34 may be spaced to engage a rod and ensure proper placement of the device 10 into a spinal cavity or space already containing a rod therewithin.

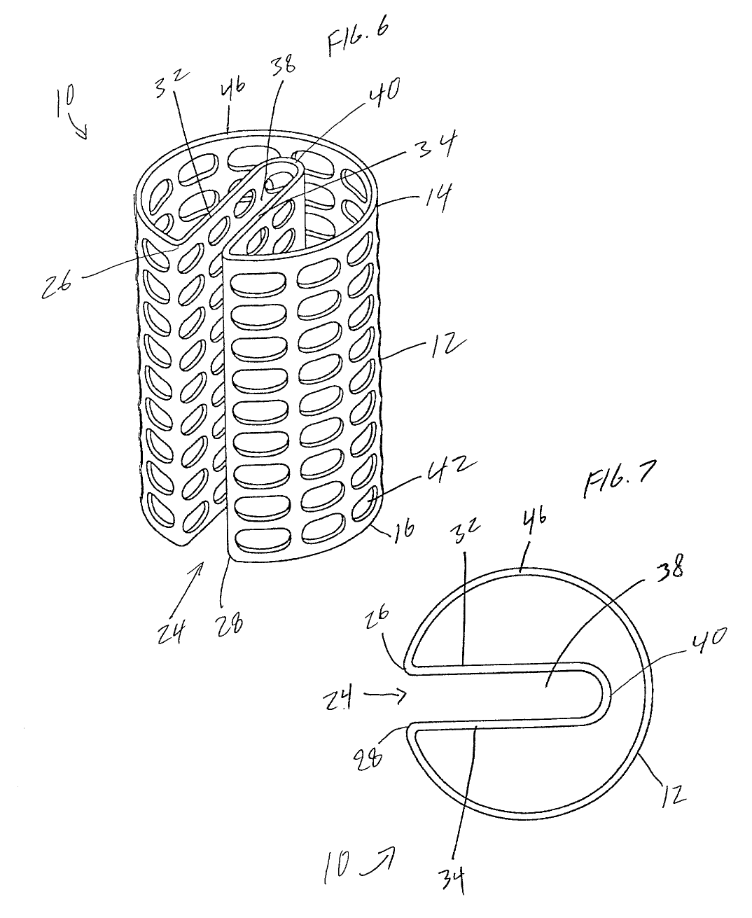

In a third embodiment shown in FIGS. 6-8, the discontinuity 24 is configured as a groove or invagination 38. The groove 38 is comprised of the inwardly extending guide walls 32 and 34 which extend inward from the edges 26 and 28. In the present embodiment however, the walls 32 and 34 are continuous being joined together at a junction 40 to form the U-shaped groove 38. Groove 38 is constructed and arranged to receive an intermedullary rod 50 (shown in FIG. 8) between the walls 32 and 34. The position of the junction 40 allows the device 10 to engage a rod 50 already in place between spinal bodies, such that the rod acts as a guide post to ensure proper positioning of the device within a spinal cavity.

In the embodiments shown in FIGS. 2-7 the longitudinal discontinuity 24 will define a space of about 5 mm to about 10 mm between the first edge 26 and second edge 28.

In the various embodiments shown in FIGS. 1-8, the body 12 of device 10 may be constructed from any biocompatible material that has sufficient rigidity and strength to at least temporarily withstand the loads imposed by body weight and the forces exerted by muscles, ligaments and tendons surrounding vertebral bodies 52 and 54 (shown in FIG. 1). Some examples of suitable materials from which the body 12 may be at least partially constructed include but are not limited to: stainless steel, titanium, nitinol, and polymer materials. Though the body 12 must be capable of withstanding load forces associated with a spinal body, the body 12 is also preferably sufficiently flexible to allow the device some degree of compressibility and flexibility in stimulate bone growth therethrough. In order to further encourage the growth and fusion of bone through and around the device 10, the body 12 may be equipped with a plurality of perforations or openings 42 extending through the body 12 to allow ingress and egress of fluids and solutions and will allow the ingrowth and through-growth of blood vessels and fibrous tissue and bony trabeculae into and through the interior space 22.

In an embodiment of the invention shown in FIG. 9, the interior space 22 may be at least partially filled with fill material 44. Fill material 44 may include a block of fill material, granules of fill material such as bioceramic beads, crushed bone or any combination thereof. Bioceramic beads are described in U.S. Pat. App. Ser. No. 09/909,668 filed Jul. 20, 2001, the entire contents of which are incorporated herein by reference. Where the device 10 includes openings 42, the openings are preferably sized to retain fill material 44 within the interior space 22, but which allows body fluids such as blood to flow therethrough.

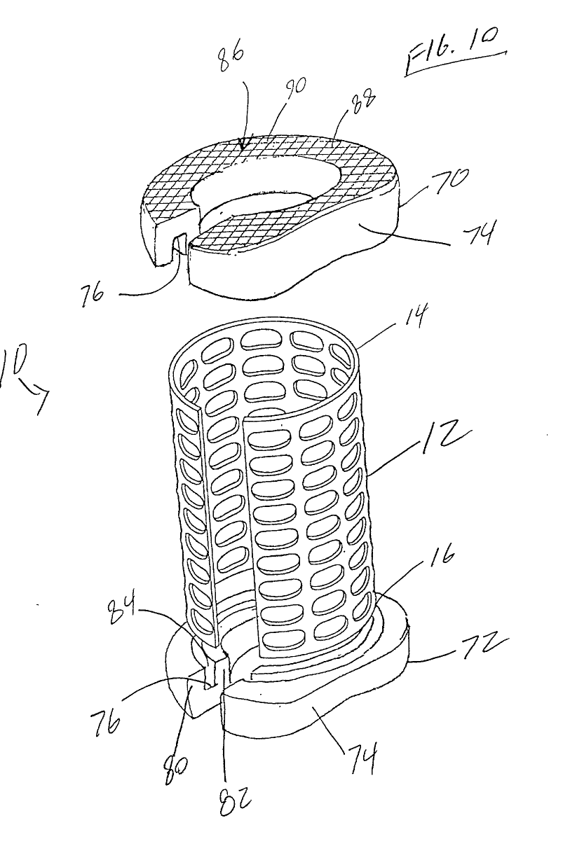

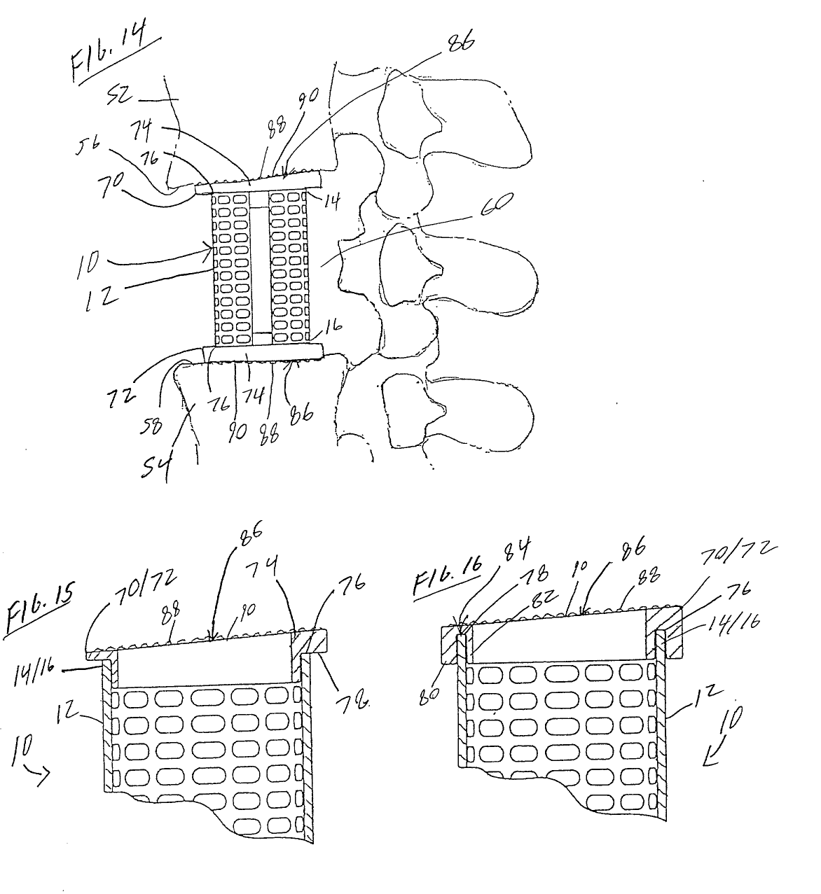

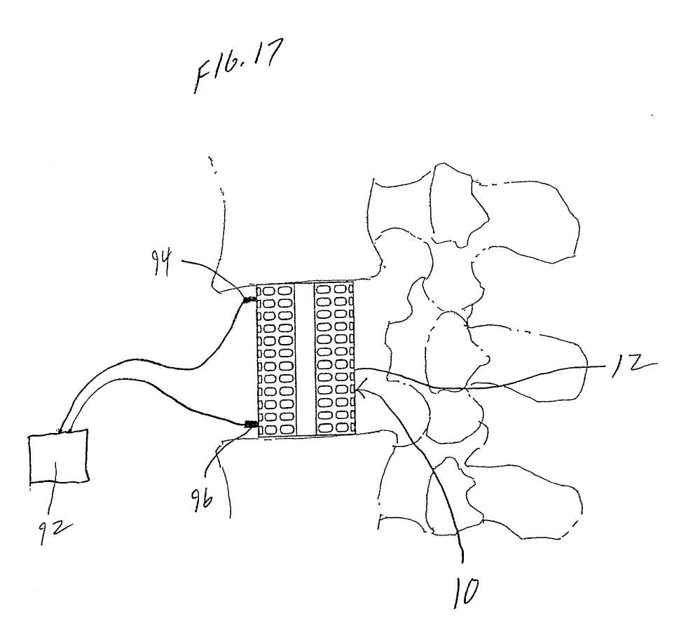

In the embodiments shown in FIGS. 2-7, the first end 14 and second end 16 are shown having substantially smooth surfaces 46. However, surfaces 46 may also be provided with surface texture in the form of ridges, protrusions or posts to engage spinal bodies adjacent thereto. Alternatively, ends 14 and 16 may be provided with end caps 70 and 72 which may be engaged to the ends 14 and 16 in the manner shown in FIGS. 10 and 14. Examples of end caps are shown in FIGS. 10-16.

End caps 70 and 72 are comprised of an end cap body 74 and include an engagement surface 76. The engagement surface 76 may be defined by an engagement lip 78 such as is shown in FIG. 15 or alternatively as a pair of engagement members 80 and 82 which define a groove 84 such as is shown in FIGS. 10, 12 and 16. Other configurations of engagement surfaces may be utilized.

As may be seen in FIG. 14, end caps 70 and 72 may be configured to have a variety of shapes to allow the second engagement surface 86 of the end cap to engage the surface 56 and 58 of a spinal body 52 and 54, regardless of the relative angle between the device 10 and spinal body 52 or 54. In addition, the end caps 70 and 72 may be provided in various sizes to allow a body 12 of a standard size and shape to be used in a wide variety of sizes of intervertebral spaces 60. In the various embodiments shown herein the end caps 70 and 72 may have a diameter equal to or larger than the diameter 100 (illustrated in FIG. 1) of the body 12.

In an alternative embodiment of the invention, multiple end caps may be stacked upon each other, at either or both ends 14 and 16 of the body 12, to effectively extend the length 98 of the device 10, thereby ensuring that a device of a given size may be utilized in an intervertebral space 60 which is longer than the body 12. If a body 12 of a given size is too large to fit into an intervertebral space 60, the body 12, may be cut to a useable size by a suitable cutting tool (not shown).

As may be seen in the various embodiments shown in FIGS. 10-16 an end cap 70 or 72 may be equipped with an engagement surface 86 having engagement members 88, which may be characterized as protrusions, ridges, posts, engagement teeth, or other forms of surface features.

The embodiment of the end cap 70 or 72 shown in FIG. 11 is configured to engage an end 14 or 16 of the device 10 shown in FIGS. 2, 3 and 10.

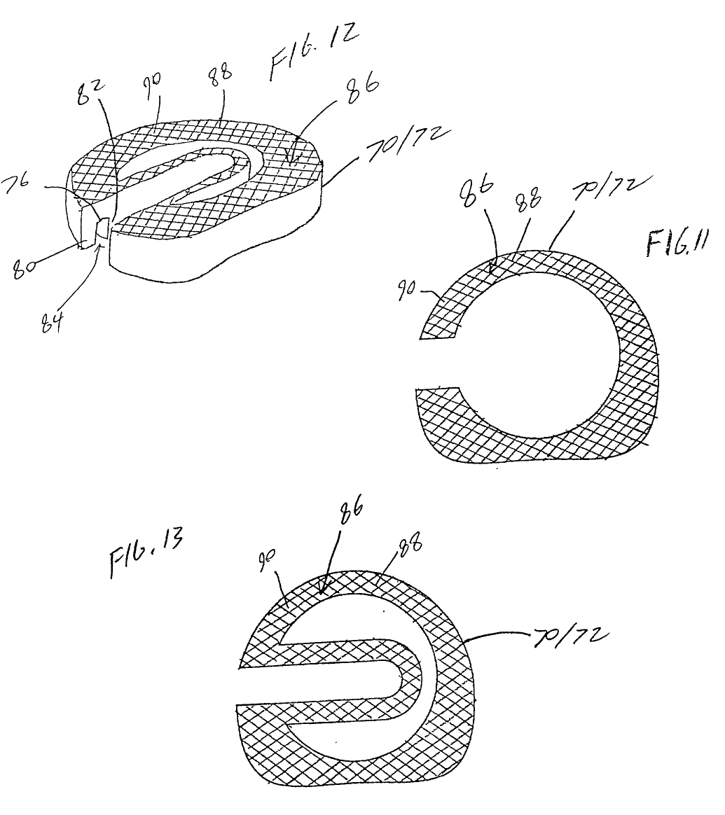

The embodiment of the end cap 70 or 72 shown in FIGS. 12 and 13 is configured to engage an end 14 or 16 of the device 10 shown in FIGS. 6 and 7.

In the various embodiments shown, end caps 70 and 72 may also include perforations or openings 90 to encourage bone growth therethrough.

To further encourage bone growth through the device 10, the device 10, such as is shown in FIG. 17, may be electrically connected to an electrical stimulation device 92 by terminals 94 and 96. Electrical stimulation device 92 provides an electric current to the device 10 and any fill material contained therein. The electric current may assist in stimulating bone growth through the device 10.

In addition to being directed to the specific combinations of features claimed below, the invention is also directed to embodiments having other combinations of the dependent features claimed below and other combinations of the features described above.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

1. A device for implantation into a mammalian spine, the device comprising:
a body, the body having a first end and a second end, the first end constructed and arranged to engage a lower surface of a first spinal body when the body is implanted into an intervertebral space of the mammalian spine, the second end constructed and arranged to engage an upper surface of a second spinal body when the body is implanted into the intervertebral space of the mammalian spine, the body having an exterior surface and an interior surface, the interior surface defining an interior space, the body further having a first edge and a second edge, the first edge and the second edge defining a longitudinal discontinuity extending from the first end of the body to the second end of the body.

2. The device of claim 1 wherein the longitudinal discontinuity is an opening defined by the first edge and the second edge, the interior space being in fluid communication with an exterior through the longitudinal opening.

3. The device of claim 2 wherein the opening is sized to allow an intermedullary rod to pass from the exterior to the interior space when the device is implanted the intervertebral space of the mammalian spine.

4. The device of claim 2 wherein the first edge and the second edge are spaced apart by less than about 10 mm.

5. The device of claim 2 wherein the body further comprises a first interior wall and a second interior wall, the first wall extending from the first edge into the interior space, the second wall extending from the second edge into the interior space, the first wall and the second wall forming a slot.

6. The device of claim 5 wherein the first wall and the second wall are substantially parallel to one another.

7. The device of claim 5 wherein the slot is sized to allow an intermedullary rod to pass therethrough from the exterior to the interior space when the device is implanted the intervertebral space of the mammalian spine.

8. The device of claim 1 wherein the longitudinal discontinuity comprises a groove, the groove being defined by a first interior wall extending inwardly from the first edge and a second interior wall extending inwardly from the second edge, the first wall and the second wall and the second wall being engaged to one another at a junction point.

9. The device of claim 8 wherein the groove is U-shaped.

10. The device of claim 8 wherein at least a portion of the first wall and the second wall are spaced apart to allow an intermedullary rod to pass between the first wall and the second wall into the groove.

11. The device of claim 10 wherein the at least a portion of the first wall and the second wall are constructed and arranged to engage the intermedullary rod when the device is implanted the intervertebral space of the mammalian spine.

12. The device of claim 1 wherein the interior space is constructed and arranged to receivingly retain fill material therein.

13. The device of claim 12 wherein the fill material is selected from at least one member of the group consisting of: bone, bone graft, bone substitute, bioceramic beads and any combination thereof.

14. The device of claim 13 wherein the fill material includes at least on member of the group consisting of: at least one type of protein, at least one type of hormone, at least one type of enzyme, at least one type of genetic material, at least one type of viral material, at least one bone formation stimulant, and any combination thereof.

15. The device of claim 12 wherein the body is constructed and arranged to be in electrical communication with an electrical stimulation device, the electrical stimulation device constructed and arranged to electrically stimulate bone formation through the device when the device is implanted into the mammalian spine.

16. The device of claim 15 wherein the electrical stimulation device is further constructed and arranged to electrically stimulate bone formation through the fill material.

17. The device of claim 1 wherein the body further comprises a plurality of openings, the plurality of openings providing fluid communication between the interior space and an exterior.

18. The device of claim 1 wherein the body further defines a cross-sectional shape, the cross-sectional shape being generally analogous to a cross-sectional shape of a normal human vertebral body.

19. The device of claim 1 wherein at least one of the first end and second end have a smooth surface.

20. The device of claim 1 wherein at least one of the first end and second end have a textured surface.

21. The device of claim 1 further comprising at least one end cap, the at least one end cap having a first engagement surface for engaging at least one of the first and second end of the body, and a second engagement surface for engaging at least one of the upper surface and lower surface of at least one of the first spinal body and the second spinal body.

22. The device of claim 21 wherein the second engagement surface is flat relative to at least one of the upper surface and lower surface of at least one of the first spinal body and the second spinal body.

23. The device of claim 21 wherein the at least one end cap has an angular orientation relative to at least one of the upper surface and lower surface of at least one of the first spinal body and the second spinal body.

24. The device of claim 21 wherein the second engagement surface of the at least one end cap has a textured surface.

25. The device of claim 21 wherein the at least one end cap further comprises a plurality of end caps, the plurality of end caps being constructed and arranged to be stacked upon each other.

26. The device of claim 21 wherein the at least one end cap has a diameter wider than the body.

27. The device of claim 1 wherein the body is at least partially constructed from a biocompatible material.
